(19) United States
(12) Patent Application Publication
(10) Pub. No.: US 2004/0053212 A1
(43) Pub. Date: Mar. 18, 2004

(54) Tissue treatment

(76) Inventors: Jacobus Stark, Zm Rotterdam (NL); Edith Magda Lucia Geijp, Pijnacker (NL)

Correspondence Address: Kate H Murashige, Morrison & Foerster, Suite 500, 3811 Valley Centre Drive, San Diego, CA, 92130-2332, US

(21) Appl. No.: 10/451,448

(22) PCT Filed: Dec. 19, 2001
(86) PCT No.: PCT/EP01/15046

(30) Foreign Application Priority Data
Dec. 20, 2000 (EP) 00204664.7

(51) Int. Cl. C12Q 1/00; A23C 3/00

(52) U.S. Cl. 435/4; 426/523

(57) ABSTRACT

The present invention describes a method of isolating or extracting fluid from a tissue comprising treating the tissue with microwaves and collecting the resulting tissue fluid.

### FIELD OF THE INVENTION

The present invention relates to novel sampling methods and apparatus to obtain large numbers of representative fluid samples from organic tissues. These fluid samples can be used for further analysis, e.g. for the detection of unwanted compounds such as veterinary drug residues in animal tissues; pesticides in fruits and vegetables or for determining the concentration of specific compounds in tissues such as the sugar content of sugar beets.

### BACKGROUND OF THE INVENTION

As most test systems to determine the concentration of specific compounds (analytes) in non liquid samples and in particular for organic tissues require fluid samples, representative fluid samples have to be obtained. Examples of such analytes are veterinary drug residues in animal tissues and residues of pesticides in vegetable tissues. Test systems for determining the content of tissue compounds, such as sugar content in sugar beets or nitrate content in vegetables, often also require fluid samples.

Controls on the presence of veterinary drug residues or diseases in slaughtered animals, consumption meat or other foods from for example cattle, pigs, poultry, sheep and fish is an important issue in the food industry. Examples of veterinary drugs are antimicrobials, antibiotics, parasiticides, growth promoters, growth inhibitors, hormones and vaccines.

Fruits and vegetables, which during or after harvesting are treated with pesticides may also contain unwanted compounds.

Veterinary drugs are administrated in cases of illness in animals. In modern farming, veterinary pharmaceuticals are sometimes also used as feed additives. These feed additives prevent illness, enhance growth or enhance feed efficiency. The compounds may be added to the feed or the drinking water of the animals. In some countries administration by injections, of for example growth hormones, is allowed.

Treatment of animals may lead to the presence of residues in muscle and/or organ tissues, which is not desired for consumption. Treatment of crops with compounds such as herbicides will also lead to the presence of unwanted residues.

It is well known that high concentrations of such residues in food products may form a health risk, which is undesirable from the standpoint of public health. Some pesticides and veterinary drugs are even carcinogenic. The use of some antimicrobial compounds as food additives is under discussion because human pathogenic bacteria may develop resistance to antimicrobials, due to their use as feed additives decreasing their usefulness in human medicine. Finally due to their inhibition of the starter cultures the presence of, for example antibiotics, may influence the production of processed meat products such as sausages in a negative way.

For these reasons in most countries maximum residue limits or tolerance levels for compounds such as drug residues in fresh meat/meat products and for pesticides in fruits and vegetables have been established. Some compounds are so undesirable that they may never be allowed in food products (zero tolerance).

For most drugs a withdrawal period is determined. This is the minimal period between the last treatment and the time of slaughter. During this period the residues of the veterinary drug decrease to a level below the established maximum allowable residue limit. However, even after the withdrawal period sometimes too high a concentration of the drug may still be present in the animal. This may be caused by natural individual differences in the metabolism of animals or because of disturbance of the excretion process of the drug due to illness. Finally withdrawal periods may not have been applied correctly. Treatment of crops, fruits and vegetables with e.g. pesticides is restricted by regulation in a comparable way and similar problems exist.

Over the years in many publications the presence of unacceptably high concentrations of veterinary drug residues in food products, such as raw meat or meat products have been described. Some compounds form an even more severe risk for human health due to their carcinogenic properties. Concerns of, for example allergic reactions, effects on the intestinal flora and development of resistance of human pathogens have also been described.

In most countries the legislation concerning veterinary drug residues in food products is maintained by using an official control program. Mostly governmental institutes examine a certain percentage of the slaughtered animals and/or consumption meat on the presence of veterinary drug residues or diseases. Slaughterhouses or supermarkets may also examine raw meat, meat products and organs such as kidney and liver.

Over the years many detection methods to determine the presence of unwanted compounds such as veterinary drug residues or herbicides in food products have been developed. For the detection of residues of antimicrobial compounds, microbial growth inhibition assays, impedance metric methods, immuno assays, receptor-enzyme binding assays, enzymatic calorimetric assays, mass spectrometry and chromatographic methods such as HPLC are examples of detection methods used in practise. Several methods described above can also be used for the detection of other compounds such as hormones. For most of these tests systems fluid samples are required.

For all known test methods, the quality of the sample will always be of great influence on the reliability of the results. To determine the concentration of analytes such as veterinary drug residues in animal tissues a representative sample has to be examined. For most test systems a representative fluid sample is required. In cases of a liquid, for example milk or urine, hardly any problems in sampling will occur. Normally the sample is always representative, because it is identical to the product which has to be examined. Therefore obtaining a representative sample from a liquid product is very easy. Mostly the liquid to be tested is simply added to the test system. If the test is executed following the instructions of the producer the results should be reliable.

However, in case of non-liquids it is far more difficult to obtain representative samples for further examination. Examples of non-liquids are animal tissues, fruits and vegetables. Mostly a representative fluid sample obtained from the tissue is required for further examination. Moreover for laboratories, where many samples per day have to be examined (e.g. governmental control laboratories), methods are required which can obtain in a reproducible manner, large numbers of representative fluid samples from tissues such as animal tissues.

To obtain fluid from animal tissues at present paper discs or swabs are brought into contact with the tissue for a certain period of time leading to absorption of tissue fluid into the swab of disk. For the detection of antimicrobial residues tissue fluid can also be obtained by placing pieces of tissue, e.g. pieces of meat or kidney, directly onto agar assay plates seeded with a selected micro-organism of a microbial inhibition assay. Mostly the slices are prepared from frozen tissue samples. Liquid from the tissue will diffuse into the agar. These methods are commonly used in today's practice.

Muscle or kidney tissues are examined for the presence of veterinary drug residues using these methods. Sometimes liver tissue is examined.

Paper discs or swabs can be saturated with tissue juice by using the following method: in the case of kidneys an incision is made into the kidney pelvis and paper disks or alternatively cotton swabs are inserted into the organ and left there for approximately 30 minutes. The paper disks are saturated with tissue fluid. One of the main disadvantage of this method is the amount of available tissue fluid in the kidneys. Sometimes kidneys are “dry”, for example kidneys of goats or kidneys of other individual animals. Further the amount of liquid on the paper disc or swab is not standardized and may vary from case to case, which may lead to false positive or false negative results and poor reproducibility. As the quantity and composition of the sample is not always standardized, e.g. a kidney sample obtained using this method can contain variable concentrations of pre-urine, tissue fluid and blood. Also by obtaining the samples using these methods duplos are not always comparable, since every sample is obtained separately. Another disadvantage of this method is that a lot of handling is required, therefore it is very time consuming and expensive and advanced laboratory facilities are required.

To determine the presence of antimicrobial residues using microbial inhibition assays the paper disks or swabs containing tissue fluid can be placed on agar test plates seeded with a selected micro-organism, e.g. a Bacillus species. This test micro-organism has a certain sensitivity to antimicrobial compounds. After incubation of the plates the size of the inhibition zone around the paper disk or swab indicates if inhibitory substances were present. A disadvantage of this method occurs when testing drug concentrations close to the sensitivity of the test system. In such cases the limited amount of liquid in the paper discs or swabs out of which the drug can be released by diffusion to the test agar may lead to false negative results.

In case where pieces of meat or kidney are directly placed on agar plates it is clear that the amount of liquid which diffuses from the tissue into the agar cannot be controlled. The amount of liquid, which will diffuse from the tissue into the agar, depends on many factors, such as the contact between the tissue and the agar, the animal species (e.g. poultry, cattle, pig), the type of tissue, the size of the sample, the temperature of the tissue, the way the sample is handled, the age of the sample, etc. It is easy to understand that such methods will give unreliable and unreproducible results.

Until now no methods have been available to obtain representative liquid samples from a large numbers of tissues for further analysis.

It is quite apparent that up to now results of monitoring the presence of analytes such as veterinary drug residues in slaughter animals greatly depend on the sampling method used. The known methods are not reproducible or reliable and/or are not suitable to obtain large numbers of samples and sometimes they may even result in unjust penalties being given to farmers. In the case of false negative results food products containing an unacceptable concentration of veterinary drug residues will end up into the food chain, which is unacceptable from a public health point of view.

Veterinary drug residues which might be present in slaughter animals are for example antimicrobial compounds. Many test methods, such as microbial growth inhibition assays, have been developed for detecting antimicrobial drug residues in animal tissues. Examples of antimicrobial drugs are Beta-lactams e.g. penicillin, ampicillin and amoxicilin ; sulfonamides e.g. sulfadiazine and sulfamethazine; tetracyclines e.g. oxytetracycline and chlortetracycline ; aminoglycosides e.g. streptomycin and gentamycin; macrolides e.g. tilmicocin and lincomycin cephalosporins e.g. ceftiofur; quinolones e.g. enrofloxacin and flumequine.

Microbial growth inhibition assays for the detection of antibacterial compounds are widely used in the food industry. The principle of such test systems is that when an antibacterial compound is present in a concentration sufficient to inhibit the growth of the microorganism present in the test system the colour of an indicator, usually a redox- or acid-base indicator, will remain the same. When no inhibition occurs the test organism will grow and produce acid or reduced metabolites which will change the colour of the indicator. The known test methods often include an agar medium seeded with a suitable test organism, preferably a thermophilic strain of Bacillus. The test organism, the indicator, nutrients, optionally substances to change (preferably improve) the sensitivity to certain antimicrobial compounds in a positive or a negative way, are added into an optionally buffered agar solution. The agar solution is allowed to solidify to form the agar medium in such a way that the test organism stays alive but cannot multiply because of a low (storage) temperature. Alternatively non-thermophilic strains of Bacillus, Streptococcus or E.coli can be used. In that case preferably the nutrients have to be added shortly before incubation to the test system e.g. together with the sample, for example by adding a nutrient tablet. Alternatively also the micro-organism or the indicator may be added as a separate source to the test system, e.g. as a tablet. Examples of commercial microbial growth inhibition assays are Premi®test, Delvotest® and BR-Test® These commercial tests are produced by DSM Food Specialties, Delft The Netherlands. Premi®Test is a broad spectrum microbial screening test for the detection of antimicrobial substances in animal tissues. Premi®Test is especially developed for the detection of antimicrobial residues in animal tissues. Delvotest® is mainly used for the detection of antimicrobial residues in milk.

Premi®Test is based on inhibition of the growth of Bacillus stearothermophilus, a spore forming bacterium very sensitive to many antimicrobial compounds. A standardised number of spores is imbedded in an agar medium with selected nutrients and the acid-base indicator Bromocresol purple. After adding approximately 100 μl of the fluid sample, for example meat juice, the test is heated to 64° Celsius, e.g. by placing the test ampoules in a water bath or an incubator. The test is incubated for 2.30-3.30 hours. In cases where no antimicrobial compounds are present in concentrations sufficient to inhibit growth, the Bacillus spores will germinate and multiply. Acid will be formed, which makes the indicator change colour from purple to yellow. When antibacterial compounds are present above the detection level no growth will take place and the colour of the test will remain purple.

### SUMMARY OF THE INVENTION

The present invention discloses apparatus and methods to obtain representative fluid samples from non-liquid samples. Typically tissues to routinely detect analytes therein or to determine the concentration of certain compounds (analytes) in large numbers of non-liquid tissues such as animal tissues or plant tissue such as fruits or vegetables.

Accordingly the present invention provides a method of isolating or extracting fluid from a tissue comprising treating the tissue with microwaves and collecting the resulting tissue fluid.

The present invention provides a sampling method and apparatus to obtain representative fluid samples from such tissues. With the sampling method and apparatus large numbers of tissue samples can be treated at the same time to obtain fluid samples for further testing in a reproducible manner. This makes the analysis of multiple samples more economic and less time consuming.

More particularly, the present invention describes methods and apparatus to obtain fluid samples from animal and plant tissues. Surprisingly the present inventors have found that the sampling methods of the invention lead to a sufficient quantity and representative quantity of tissue fluid (sample) of which the analyte concentration of for example veterinary drug residues is representative for the concentration of this analyte in the tissue from which the sample is obtained.

Surprisingly the samples obtained from animal and plant tissue using the present method and apparatus were found to be representative of the tissue they are derived from. Therefore when the invention is applied to detect analytes, such as for example antimicrobial residues, false positive results are less likely to occur.

In one aspect, the present invention provides an apparatus and method to obtain representative fluid samples from large numbers of tissues, for example muscle or organ tissue. Of course by using the method and apparatus of the invention fluid samples can also be obtained from plant tissues, such as fruits and vegetables.

Accordingly, the present invention provides a device which comprises:

a first container having at least one hole or outlet means present in the bottom and/or sides of the first container;

a second container; and

a compressor or means to apply pressure

where the device is constructed in such a way that it is able to squeeze or compress a sample present in the first container, and hence releases, forces or separates fluid from the sample where the first container is constructed in such a way that some or all of the fluid separated from the sample will pass through the hole or outlet into the second container.

The present invention also provides for the use of the device of the invention to squeeze or compress a sample, preferably a tissue, more preferably an animal tissue to produce a liquid.

The inventors also described means and methods to transport the fluid sample from the apparatus to the test system to be used for further analysis, in particular to detect veterinary drug residues such as antimicrobial compounds or hormones.

Preferably, the device of the invention may form part of a test system.

### DETAILED DESCRIPTION OF THE INVENTION

The Tissue Sample

The present invention may be applied to any non-liquid sample which has some fluid content.

Typically, the invention will be applied to animal, plant or fungal tissues but may also be applied to other non-liquid items such as foodstuffs. Examples of animal tissues which may be analysed include those of pigs, sheep, poultry and beef. Examples of plant tissues include sugar beet, fruit, potato, vegetables and other commercially cultivated plants. Generally by tissue it is meant any non-liquid sample comprising cells.

A representative piece of tissue can be obtained by any method known in the art, mostly a knife will be used. However also other tools such as cutters or drills can be used. For further processing by using the apparatus disclosed in this invention and to obtain a sufficient amount of fluid sample, the piece of tissue has to be of a certain size, preferably the piece of tissue has a size of more than 0.1 cm³, preferably more than 1.0 cm³ more preferably more than 5 cm³. Typically the tissue has a volume of from 0.1 to 20 cm³, preferably from 0.2 to 5 cm³ more preferably from 0.5 to 10 cm³, most preferably of from 1 to 8 cm³. Although it is not necessary that the pieces of tissue to be examined all have exactly the same size, it is clear that the amount or size of the pieces of tissue preferably are comparable. This can be achieved by using a knife, but sometimes it might be easier to use a cutter or drill, with which more or less standardized and uniform pieces of tissue can be obtained. To take into account variations in size the samples may be weighted prior to fluid extraction.

Although the device and methods of the invention will primarily be used to analyse samples that are for consumption they may also be used for analysing samples from biopsies or post mortem samples in order to diagnose or detect disease or infection.

Pre-Treatment of Animal Tissues by Using a Microwave

To obtain a sufficient amount of liquid by squeezing for some tissues and in particular some animal tissues first a pre-treatment of the tissue by heating is advantageously applied, preferably using a microwave.

Surprisingly it has been demonstrated in the present invention that pieces of tissue and in particular animal tissue which have been pre-treated using a microwave oven are easily squeezed to give a sufficient amount of sample fluid. Preferably, the pieces of plant or animal tissue are placed in containers, which are placed in the microwave. The microwave is closed and turned on. In one embodiment of the invention the microwave may from part of the device of the invention. The exact time/capacity treatment required should be selected to pre-treat the animal tissue in such a way that the compounds (analytes) to be detected are not inactivated and that sufficient liquid can be obtained by squeezing. Preferably a treatment of from 10 to 300 seconds at a capacity of from 50 to 500 Waft, more preferably from 30 to 150 seconds at a capacity of from 60 to 200 Watt per 60 g of animal tissue is applied. In general a longer treatment with a lower power (Watt) is preferred over a short microwave treatment at a high power. Of course the exact figures depend on many factors, e.g. the heat stability of the analyte to be detected, the type of microwave and the size, type and temperature of the tissue samples. Any microwave known in the art, which can fulfil such requirements, can be used. For example, in case antibiotics have to detected the temperature has to kept below 80° C., preferably below 70° C., more preferably below 60° C. and even more preferably below 50° C. in the sample. For example a microwave treatment is chosen which results in a temperature of from 35 to 50° C., preferably from 40 to 50° C., more preferably from 45 to 50° C. of the animal tissue. In embodiment of the invention where microbial content in the fluid harvested is to be analysed microwave treatment is to be avoided or kept to a level which does not inactivate the microbial to be quantified.

One aspect of the invention is the use of a microwave for the pre-treatment of animal tissues to obtain sufficient tissue fluid required for further examination, e.g. for the presence of veterinary drug residues. The invention also includes samples of fluid obtained by treating animal tissue with a microwave treatment followed by squeezing the treated tissue. Plant tissues may be prepared in a similar manner.

Description of the Container

Once the samples have been isolated, typically they are placed in a container. The microwave treatment may be carried out on the sample in the container or prior to its addition to the container.

According to another aspect of the invention, containers are therefore included, which makes it feasible to pre-treat more samples, e.g. from 2 to 500, preferably from 4-200, more preferably 10-50, at the same time. Preferably the containers are grouped in a matrix. Preferably these containers (1, 2 and 1′, 2′) are constructed in such a way, that they can be used directly in the next step (squeezing) described in the present invention.

The containers have a size large enough to place a piece of tissue in it, e.g. 4×3×3 cm (l×w×h, i.e. length by width by height). The container can be constructed from any suitable material known in the art, for example plastics or ceramics. In case where a microwave pre-treatment is given, materials are used which are suitable for use in a microwave in other words non-metallic materials are used. Preferably at least two kind of different containers are used. The first container (1 and 1′), wherein the piece of tissue (4 and 4′) is placed, has at least one hole or channel from the container. Holes might be present in the bottom and/or in the sides of the first container and in particular in the lower part of the sides. The diameter of the holes can be from 0.1 to 0.8 cm, preferably from 0.2 to 0.7 cm, more preferably from 0.2 to 0.6 cm. The holes will let pass the tissue fluid separated from the tissue into a second container. The first container with holes is typically placed above a second container (2 and 2′) or joined by means to allow fluid to pass or flow to the second container. The first and second container are constructed in such a way that the tissue liquid from the first container is collected in the second container, for example said first container fits within the second container. Alternatively a tube or a conduit may allow the passage of the fluid between the containers. In general, the second container does not contain holes. An example of such containers is presented in FIG. 1. A filter or mesh may be present, through which the tissue fluid passed in its journey form one chamber to the other. Such a filter or mesh may prevent or reduce the passage of solid particles. Of course any other containers having the same functions are included in this invention.

Obtaining Tissue Fluid by Using the Device of the Invention

In case where multiple compressors are interrelated connected together (e.g. in a matrix form) with interrelated connected series of first and second containers more then one piece of tissue can be squeezed or compressed at the same time. Preferably from 1 to 200, more preferably from 5 to 50 even more preferably from 5 to 25 pieces of tissue can be squeezed at the same time.

In this example the containers described above containing the optionally pre-treated pieces of tissue are placed in the device. A sufficient amount of tissue juice is obtained by simply pulling the lever down. The juice is collected in the second container (s) and is available for further examination. Of course tissue fluid from, e.g. animal tissues or tissue from plant origin, such as fruits and vegetables, can also be obtained by using said method. Generally the device will squeeze, compress or exert pressure on the sample.

Squeezing of the tissue by using the device presently disclosed gives a sufficient amount of representative fluid sample for further analysis. In the case of animal tissues veterinary drug residues, e.g. hormones or antimicrobial compounds such as antibiotics, these analytes are not inactivated by the squeezing or the pre-treatment using the microwave method. Drugs such as beta-lactam like pennicilin, ampicillin and amoxicilin; sulfonamides such as sulfadiazine and sulfamethazine; tetracyclines such as oxytetracyline and chlortetracyline; aminoglycides such as streptomycin and gentamycin; macrolides and quinolones may be detected. By using these methods substantial damage to the animal tissue will not occur, therefore negative effects of for example natural inhibiting compounds (from the animal tissue) which might disturb the antimicrobial residue tests were not seem to occure.

The methods and apparatus described in this invention are able to give representative fluid samples of animal and plant tissues such as raw meat, consumption meat, kidneys, liver, fruits and vegetables. For the first time it is possible to obtain in a predictable manner fluid samples form a large number of tissue types and a large number of samples in one step which can be used for further testing on e.g. analytes like antimicrobials, hormones and pesticides can be detected in this way or can be used to test for diseases for example BSE, scrapie, virus, fungal, bacterial and nematode infections and pathogenic bacteria. Environmental pollutants may also be detected. These samples can also be used to determine the concentration of specific compounds, e.g. the sugar content of sugar beets or the nitrate content of vegetables.

Transport the Fluid Sample to the Test System

Due to the squeezing and compression the fluid sample will be collected in the second container(s) described above. Subsequently, the fluid sample can be brought directly to the test system or alternatively to for example a tube to collect the sample for further analysis by using e.g. a pipette or for freezing until analysis can be carried out. Alternatively the liquid may immediately drip into for example a tube or directly into the test system, for example an ampoule. In this case the tube or test system is in fluid contact of or connected with the second container or forms part of the second container. To prevent an uncontrolled dripping of the fluid into the tube or test system a cap or a dropper can be included. By using the dropper the required amount of liquid can be limited and recorded. Further to prevent contamination of the liquid with solid particles also a constriction or filter can be placed, for example at the bottom of the first or second container. Such a means forms part of this invention. Before analysis serial dilutions of the fluid may be made to help more accurately determine the level or concentration of the analyte.

Integrated Sampler/Test

Advantageously the described methods to obtain representative fluid samples of animal or plant tissue can be optimally integrated with test systems such as antimicrobial residues tests, e.g. a microbial inhibition assay. The test system to e.g. the presence of veterinary drug residues can form part of the apparatus to obtain the fluid.

Verious techniques such as ELISA, masspectroscopy, electrophoresis, PCR, test-strips, cell culture assays, antibody based tests and mutagenicity tests such as the Ames test may also be used to analyse the sample.

A test to detect analytes in the sample fluid, can be combined with the second container in which the fluid sample is collected. The second container can be constructed in such a way, ti; at at least one test is integrated in the second container in which the fluid sample is collected.

An example is the integration of antibiotic residue tests such as a microbial inhibition test. The skilled person will appreciate that other tests for analytes can be integrated in this system as well.

In the case of a test based on the use of a test medium, such as Premi®Test, the test medium can be included in the second container in which the fluid sample is collected after squeezing. In this case the second container forms part of the test device. According to another embodiment the liquid may flow from the second container into a test device containing for example the agar by using a cap or a dropper at the bottom side of the second container.

The test medium can be added, preferably in advance, which is illustrated in FIG. 2. Some examples are presented in FIG. 2. In case devices are developed as illustrated in FIG. 2 the tube like part, which contains the test medium, can optionally also be removed by breaking the tube from the second container. Said means, which are also included in this invention, can be constructed by methods well known in the art.

The test devices system can of course be any system for the detection of any analyte or compound, e.g. any veterinary drug residue or pesticides.

Also more than one test device may be constructed as integral part of the second container, which allows the possibility of obtaining for example duplo tests or detecting more compounds from one sample. In this case the second container may contain different selective media for the detection of specific groups of antimicrobial drug residues or may contain test systems for the detection of other veterinary drug residues, such as hormones. Examples of specific groups of veterinary drugs are beta lactam antibiotics, sulfonamide compounds, tetracyclines and quinolones. Various controls such as the use of known or standard samples containing set amounts of the analyte under investigation of lacking the analyte may be used.

This invention includes all variants of the apparatus or devices presently disclosed. The skilled person will appreciate that automated or computerised systems can be designed based on the apparatus described in this invention. Such systems may store and/or process the results obtained. The devices of the invention may be portable.

Squeezing

All kinds of squeezers can be used to squeeze animal or plant tissue to obtain tissue juice which can be tested in the devices of the invention. The device may squeeze one sample, but preferably squeeze two or more samples at the same time, more preferably from 2 to 100, even more preferably from 5 to 25 samples. Each sample is placed in a first container. Subsequently, the device will squeeze or compress the contents of this container. The device can be manually, electrically or pneumatically operated. In general the tissue samples are squeezed by a compressor (3 and 3′) of the device. Alternatively, other means for applying pressure may be used in place of the compressor. The compressor is constructed in such a way that fits within the first container. The compressor surface is such that the tissue cannot substantially escape besides the rinse of the compressor surface or compressor. Moreover the compressor is dimensioned in such a way that the tissue juice will not overflow the first container (1 and 1′). In a preferred embodiment the first container (1 and 1′) and/or the compressor (3 and 3′) are constructed conically as shown in FIG. 1.

Advantageously distance holder means are positioned in the device. The first distance holder means takes care that the distance between the compressor surface and the bottom of the first container can be limited. For example a minimal distance of 2 mm can be adjusted by the distance holder present on for example the compressor surface, the bottom of the first container or can be adjusted elsewhere within the device. A second distance holder means takes care that the first and second container are separated in distance, which distance is enough to prevent that the containers sticking together and also to take care that enough volume between the containers is present to receive to the tissue juice without overflow.

The second distance holder may be part of the first or second container or may be present as a means which is part of the device.

According to preferred embodiment the device is applied with a separating means which enables separation of the first container from the second container after the squeezing has taken place. The separating means preferably automatically separates these containers at the same time as the compressor is lifted or removed from of the first container.

The compressor, like the rest of the device of the invention will preferably be simple to clean and disinfect and may be sterilizable. Alternatively, the compressor and/or containers may be replaced after each use.

### LEGENDS TO THE FIGURES

FIG. 1 shows an embodiment of the device which has at least two first containers at least two second containers and at least two compressors.

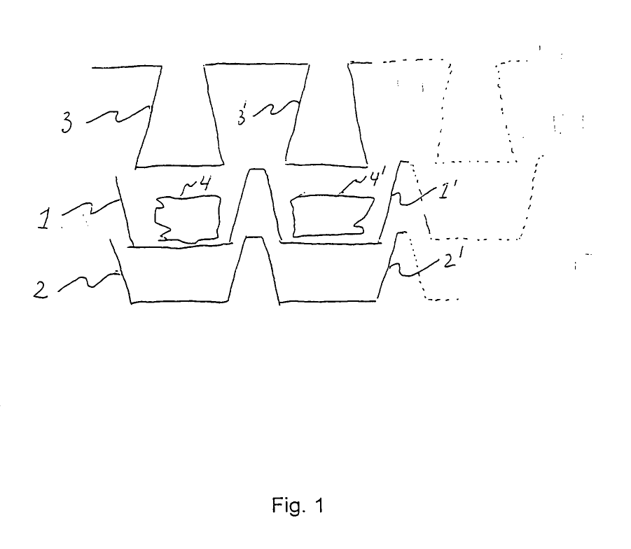

FIG. 2 shows embodiments of the second container which contains a test medium or which is connected with a further container comprising test medium.

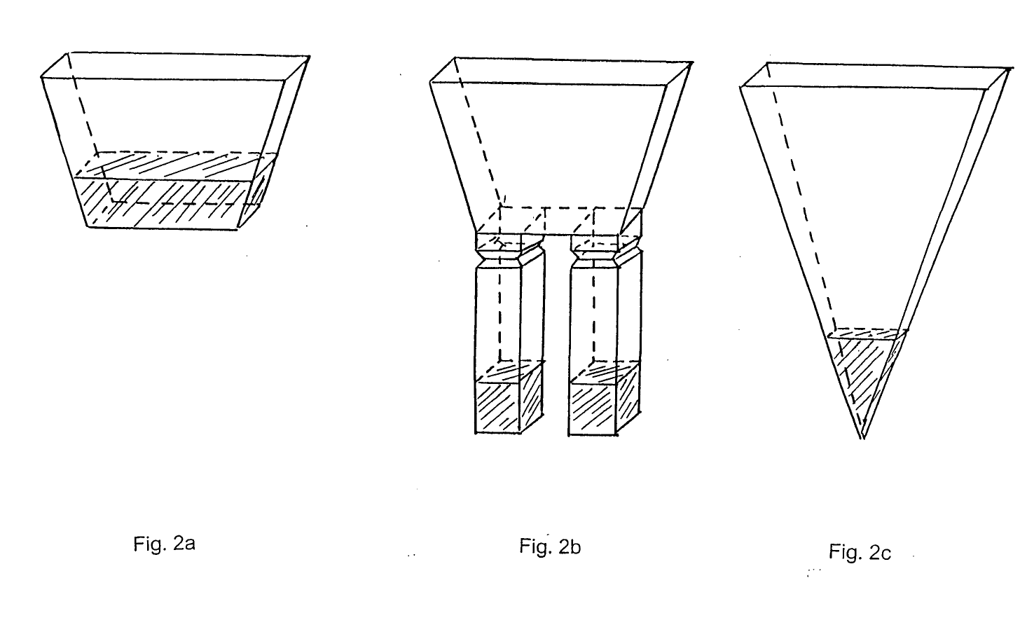

### EXAMPLE 1

Obtaining Tissue Juice from Several Animal Tissues and Examining these Liquid Samples on the Premi®Test

Fresh muscle tissue from chicken, pigs and cows and kidneys from cows were obtained from a slaughterhouse. All tissues were negative in the sense of the absence of antimicrobial residues.

Pieces of tissue of approximately 6.0 cm³ were obtained by cutting with a knife, 12 pieces of each tissue were placed in a first container of 3.5×2.5×2 cm (l×w×h) whereby 12 containers were connected to each other in two rows of 6 containers. 12 samples were pre-treated together in the microwave for 90 seconds at 90 Watt . The temperature of all the samples was for between 35 and 50° C. The container containing the pre-treated samples was then placed in the device. A fluid sample was obtained by squeezing the samples.

Sufficient liquid was obtained from each sample. The amount of liquid per sample was approximately 1 ml.

100 μl of all of the fluid samples isolated was placed on the Premi®Test, a commercial broad-spectrum microbial test for the detection of antimicrobial residues. This test was produced according to the methods described in EP 0005891 with the nutrients present in the agar.

The test was incubated following the instructions of the producer. After 165-175 minutes the colour of all tests turned from purple to yellow, indicating that no antimicrobial residues or disturbing compounds from the tissue were present. No false positive results have been found.

These results clearly demonstrate that in one step, 12 animal tissue samples can be processed for obtaining fluid samples. The fluid samples are representative, since no false positive results were found in the Premi®)Test results.

### EXAMPLE 2

Stability of Anti Microbial Compounds Pre-Treated in the Microwave.

In this experiment it is demonstrated that anti microbial residues are stable when treated using a microwave.

Liquid sample obtained from squeezing cows muscle tissue was spiked with anti microbial compounds. Amoxicillin, sulfadiazin and oxytetracycline were added to the meat juice in a final concentration of respectively 10, 100 and 400 ppb. The solutions were divided into two portions. One part was treated by microwaving whereby 12 samples were placed in 12 interconnected sample containers. The 12 samples were treated at the same time for 15 seconds at a power of 90 Watt. The other part was used as control and not microwaved.

After the treatment in the microwave the samples were diluted with blanco meat juice free from anti-microbial residues to final concentrations between 2-400 ppb.

100 μl of juice from each sample was placed on the PremiφTest, a commercial broad-spectrum microbial test for the detection of antimicrobial residues. This test was produced according to the methods described in EP 0005891 with the nutrients present in the agar. The test was incubated following the instructions of the producer. As a control meat juice without antibiotics was examined.

Tests incubated with the control samples without antibiotics turned yellow after 170 minutes. At that time the tests incubated with the spiked samples were read.

All samples with the same concentrations of antimicrobial compounds gave the same results, regardless of whether they had been microwaved or not indicating that the treatment with the microwave does not inactivate the antimicrobial compounds. More specifically: all tests containing samples with concentrations of amoxicillin at and above 6 ppb, sulfadiazine at and above 50 ppb and oxytetracycline at and above 200 ppb remained purple. All other tests turned yellow.

These results clearly demonstrate that treatment of the samples in the microwave does not inactivate the antimicrobial compounds to be analysed and also the sensitivity of the assay.

### EXAMPLE 3

Detection of Antibiotics in Positive Tissue Samples

In this experiment it is demonstrated that antimicrobial residues from positive tissue samples can be detected using the methods and apparatus of the present invention. As a control muscle and kidney tissue from a negative animal were included in this study.

Muscle and kidney samples of 6 positive calves were obtained from a control laboratory of a slaughterhouse. The samples were positive in the sense that they were already known to contain antimicrobial residues. Each sample was positive for one antimicrobial compound (namely tilmicosin, oxytetracycline, amoxicillin, .ceftiofur, sulfadiazine and gentamycin, respectively).

Fluid samples from these muscle and kidney tissues were obtained by cutting pieces of approximately 6.0 cm³ of each tissue. These samples were placed in the containers described in example 1 and pre-heated in a microwave oven. Control samples were obtained by using a garlic press.

100 μl of liquid was brought on the Premi®Test, a commercial broad spectrum microbial test for detection of antimicrobial residues. This test was produced according to the methods described in EP 0005891 with the nutrients present in the agar. The test was incubated following the instructions of the producer.

After 165 minutes the colour of both negative control samples turned from purple to yellow, indicating that no antimicrobial residues were present. All other samples remained purple, indicating the presence of antimicrobial residues.

These results clearly demonstrate that by using the methods and apparatus of the present invention (pre-treatment of the tissue sample using the microwave and followed by squeezing) fluid samples can be obtained without inactivating relevant antimicrobial compounds.

The fluid samples are representative, since no false negative results were found and Premi®Test gave results as expected.

### EXAMPLE 4

Tissue Fluid Obtained by the Several Sampling Methods

This experiment demonstrates that the microwave treatment is advantageous to obtain sufficient tissue fluid. Chicken, pork and beef tissue was cut in pieces of approximately 5 gram. Tissue fluid was obtained by using a device of the invention or a garlic press, with or without a pre-treatment using the microwave. The garlic press can be used as well with fresh as with microwave treated samples, but is found to be very labour-intensive, and can, of course, only process a single sample of one time. 12 samples were treated with a microwave for 90 seconds at 90 Watt. Tissue fluid was obtained by pressing 6 of the treated samples with the squeezer and 6 of the treated samples manually with the garlic press. As a control tissue fluid was obtained by pressing 6 samples with the squeezer and 6 samples with the garlic press. The amount of obtained tissue fluid was determined. The results clearly demonstrated that the microwave treatment is preferable to obtain sufficient tissue fluid. In case of chicken meat without pre-treatment in the microwave no fluid is even obtained by using the device of the invention.

Amount of tissue fluid obtained (gram)

| | Squeezer | | | Garlic press | | |
|---|---|---|---|---|---|---|
| | Chicken | Pork | Beef | Chicken | Pork | Beef |
| With microwave | 0.50 | 0.91 | 0.89 | 0.45 | 0.89 | 0.89 |
| treatment | | | | | | |
| Without microwave | 0 | 0.20 | 0.21 | 0.16 | 0.40 | 0.55 |
| treatment | | | | | | |

1. A method of isolating or extracting fluid from a tissue comprising treating the tissue with microwaves and collecting the resulting tissue fluid.

2. A method according to claim 1 wherein tissue is squeezed or compressed during or after the microwave treatment to obtain the tissue fluid.

3. A method according to claim 1 or 2, wherein the tissue is an animal or plant tissue.

4. A method according to any one of the preceding claims wherein the temperature of the tissue is maintained below 80° C. during the microwave treatment.

5. A method according to any one of the preceding claims wherein the tissue is treated for from 10-300 seconds at from 50-500 Watt, preferably for from 30-150 seconds at from 60-200 Watt, per 60 g of tissue.

6. A method according to any one of the preceding claims, wherein the tissue is an animal tissue.

7. A method according to anyone of the preceding claims wherein the tissue is squeezed or compressed after the microwave treatment.

8. A method according to any one of the preceding claims wherein the tissue is microwaved and squeezed or compressed in the same device.

9. A method according to any one of the preceding claims wherein the fluid obtained is tested to determine the presence, absence or concentration of an analyte.

10. A method according to any one of the preceding claims wherein the liquid is tested in the same device which squeezed and microwaved the tissue.
